(19) United States
(12) Patent Application Publication
(10) Pub. No.: US 2026/0020908 A1
(43) Pub. Date: Jan. 22, 2026

(54) MICROWAVE ABLATION PROBE COOLANT CONDUIT

(71) Applicant: Varian Medical Systems, Inc., Palo Alto, CA (US)

(72) Inventors: Justin May, Austin, TX (US); Hojjatollah Fallahi, Minneapolis, MN (US)

(21) Appl. No.: 18/778,579

(22) Filed: Jul. 19, 2024

(51) Int. Cl. A61B 18/18; A61B 18/00

(52) U.S. Cl. CPC A61B 18/1815; A61B 2018/00017; A61B 2018/00023; A61B 2018/00077; A61B 2018/00083; A61B 2018/00577; A61B 2018/1823

(57) ABSTRACT

A microwave ablation probe includes a shell extending in an axial direction and defining an inner cavity, a tip positioned on a distal end of the shell, a cable with a microwave antenna extending axially in the inner cavity, and a coolant conduit positioned radially outward of the cable defining a coolant flow path toward the tip. A distal end of the coolant conduit includes an angled end surface oriented at an angle relative to an axis of the coolant conduit.

### TECHNICAL FIELD

The disclosure relates to coolant conduits used in microwave ablation probes.

### BACKGROUND

Microwave ablation probes can be used in clinical treatments such as thermal ablation treatments. In such treatments, thermal ablation can be used to destroy undesirable tissue such as malignant cells in a body. A microwave ablation antenna can be included in the probe and be used to deliver Radio Frequency (RF) energy such as microwave energy to a target tissue to heat the target tissue and destroy the target tissue. The microwave ablation antenna can be positioned inside the ablation probe that can position the microwave ablation antenna proximate the target tissue.

In some treatments, the ablation probe is guided to the target tissue though other tissue or near to tissues or body structures that it is desirable not to damage during treatment. It is desirable, therefore, to maintain a small size of the microwave ablation antenna and/or the needle of the probe. In this manner, damage to tissues and other body structures that may be located close to the target tissue to be destroyed is minimized or prevented. It can be difficult to manufacture ablation probes that have suitably sized for thermal ablation due to the complexity of such devices and the need to position all elements so that an ablation zone can be repeatably achieved. There exists a need, therefore, for improved microwave ablation probes or needle designs that are sufficiently small, deliver known and repeatable ablation zones and can be manufactured with high quality and low cost.

### SUMMARY

The present disclosure is directed to microwave ablation probes that may include an improved coolant conduit over existing probes. The coolant conduits of the ablation probes of the present disclosure may include features to ensure that the conduits are properly positioned and continue to operate as intended in instances in which the conduit may move or be displaced after assembly. The coolant conduits of the present disclosure may also operate so that a reliable, repeatable ablation zone is produced during operation. Such features as described below are not found in existing microwave ablation probes. The coolant conduits of the present disclosure result in microwave ablation probes that do not have performance issues that may arise from inadvertent manufacturing defects that may be difficult to detect in some instances.

In accordance with some embodiments, a microwave ablation probe is provided. A microwave ablation probe may include a shell extending in an axial direction and defining an inner cavity, a tip positioned on a distal end of the shell, a cable with a microwave antenna extending axially in the inner cavity, and a coolant conduit positioned radially outward of the cable defining a coolant flow path toward the tip. A distal end of the coolant conduit may include an angled end surface oriented at an angle relative to an axis of the coolant conduit.

In another aspect, the coolant conduit includes a distal portion connected to a proximal portion, the distal portion made of a different material from the proximal portion and terminating at the angled end surface.

In another aspect, the proximal portion may be made of a metal material.

In another aspect, the distal portion may be made of a polyimide material.

In another aspect, the coolant flow path toward the tip may be located inside the coolant conduit.

In another aspect, the angled end surface may not be oriented parallel to an end surface of the tip that faces the coolant conduit in the inner cavity.

In another aspect, the distal end of the coolant conduit may be positioned at or near the antenna in the inner cavity.

In another aspect, the angled end surface may define a planar surface.

In another aspect, the angled end surface may define a pointed end of the coolant conduit.

In another aspect, the angled end surface may define a pointed end located at a center axis of the coolant conduit.

In some embodiments of the present disclosure, a coolant tube for use in a microwave ablation probe is provided. The coolant tube may include a proximal portion, and a distal portion connected to the proximal portion, wherein the proximal portion and the distal portion define a continuous coolant flow path, a distal end of the distal portion with an angled end surface.

In one aspect, the proximal portion may be made of a metal material.

In another aspect, the distal portion may be made of a polyimide material.

In another aspect, the angled end surface may define a planar surface.

In another aspect, the angled end surface may define a pointed end of the coolant conduit.

In another aspect, the angled end surface may define a pointed end located at a center axis of the coolant conduit.

In some embodiments, a microwave ablation apparatus is provided.

The microwave ablation apparatus may include an ablation console with a microwave generator, and a microwave ablation probe coupled to the ablation console. The microwave ablation probe may include a shell extending in an axial direction and defining an inner cavity, a tip positioned on a distal end of the shell, a cable with a microwave antenna extending axially in the inner cavity, and a coolant conduit positioned radially outward of the cable defining a coolant flow path toward the tip. A distal end of the coolant conduit may include an angled end surface oriented at an angle relative to an axis of the coolant conduit.

Further areas of applicability will become apparent from the description provided herein. The description and specific examples in this summary are intended for purposes of illustration only and are not intended to limit the scope of the present disclosure.

### BRIEF DESCRIPTION OF THE DRA WINGS

The features and advantages of the present disclosures will be more fully disclosed in, or rendered apparent by the following detailed descriptions of example embodiments. The detailed descriptions of the example embodiments are to be considered together with the accompanying drawings wherein like numbers refer to like parts and further wherein:

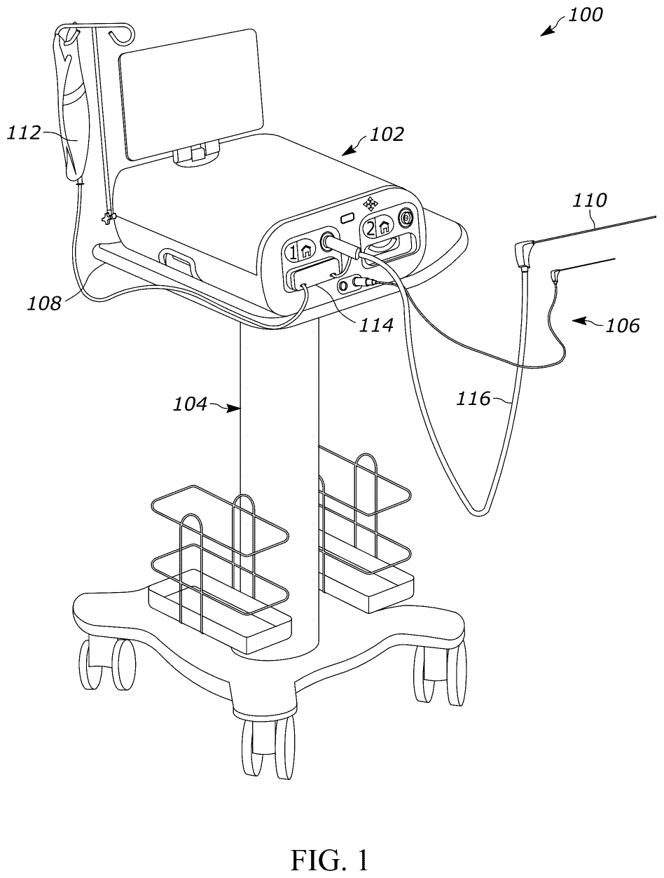

FIG. 1 is an isometric view of an example microwave ablation apparatus in accordance with some embodiments of the present disclosure.

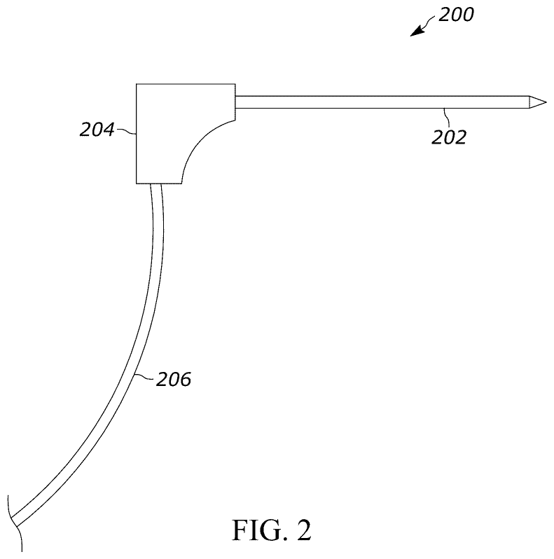

FIG. 2 is an example microwave ablation probe in accordance with some embodiments of the present disclosure

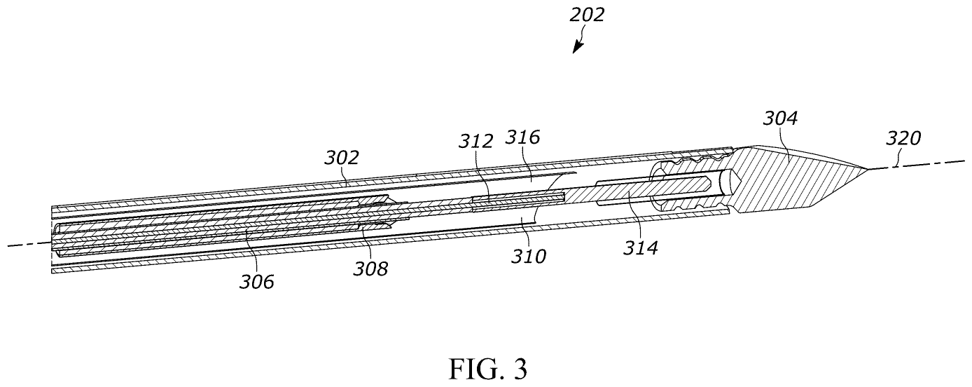

FIG. 3 is a side sectional view of an example coolant conduit assembled in a microwave ablation probe of the present disclosure.

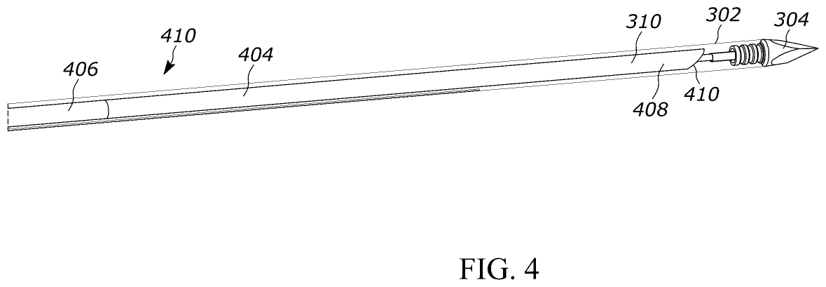

FIG. 4 is a side view of the example microwave ablation probe of FIG. 3 with the shell transparent to show inner elements of the probe.

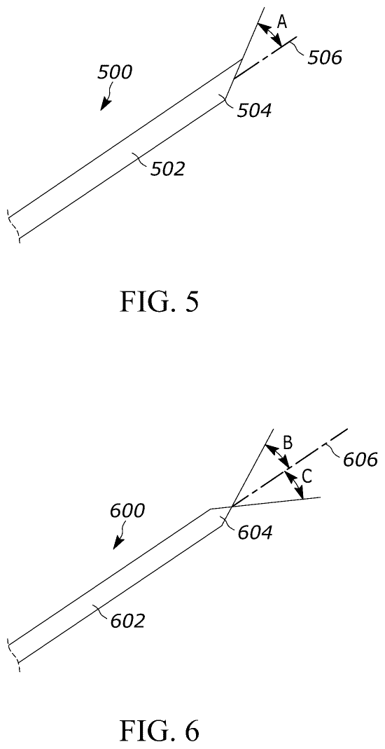

FIG. 5 is a side view of an example coolant conduit in accordance with some embodiments of the present disclosure.

FIG. 6 is a side view of another example coolant conduit in accordance with some embodiments of the present disclosure.

### DETAILED DESCRIPTION

The description of the preferred embodiments is intended to be read in connection with the accompanying drawings, which are to be considered part of the entire written description of these disclosures. While the present disclosure is susceptible to various modifications and alternative forms, specific embodiments are shown by way of example in the drawings and will be described in detail herein. The objectives and advantages of the claimed subject matter will become more apparent from the following detailed description of these exemplary embodiments in connection with the accompanying drawings.

It should be understood, however, that the present disclosure is not intended to be limited to the particular forms disclosed. Rather, the present disclosure covers all modifications, equivalents, and alternatives that fall within the spirit and scope of these exemplary embodiments. The terms “couple,” “coupled,” “operatively coupled,” “operatively connected,” and the like should be broadly understood to refer to connecting devices or components together either mechanically, electrically, wired, wirelessly, or otherwise, such that the connection allows the pertinent devices or components to operate (e.g., communicate) with each other as intended by virtue of that relationship.

In the present disclosure the singular forms “a,” “an,” and “the” include the plural reference, and reference to a particular numerical value includes at least that particular value, unless the context clearly indicates otherwise. When values are expressed as approximations, by use of the antecedent “about,” it will be understood that the particular value forms another embodiment. As used herein, “about X” (where X is a numerical value) preferably refers to +10% of the recited value, inclusive. For example, the phrase “about 8” preferably refers to a value of 7.2 to 8.8, inclusive. Where present, all ranges are inclusive and combinable. For example, when a range of “1 to 5” is recited, the recited range should be construed as including ranges “1 to 4”, “1 to 3”, “1-2”, “1-2 & 4-5”, “1-3 & 5”, “2-5”, and the like. In addition, when a list of alternatives is positively provided, such listing can be interpreted to mean that any of the alternatives may be excluded, e.g., by a negative limitation in the claims. For example, when a range of “1 to 5” is recited, the recited range may be construed as including situations whereby any of 1, 2, 3, 4, or 5 are negatively excluded; thus, a recitation of “1 to 5” may be construed as “1 and 3-5, but not 2”, or simply “wherein 2 is not included.” It is intended that any component, element, attribute, or step that is positively recited herein may be explicitly excluded in the claims, whether such components, elements, attributes, or steps are listed as alternatives or whether they are recited in isolation.

Turning now to FIG. 1, an example microwave ablation apparatus 100 is shown. In this example, the ablation apparatus 100 includes an ablation console 102, and an ablation probe 110. The ablation console 102 may be portable, in some examples, and may be positioned on a platform 108 of stand 104 to allow the console 102 to be easily moved and positioned as desired during treatment. The console 102 may include a microwave generator that may provide a power signal that is sent to an antenna in the needle of the ablation probe 110. The power signal may be provided to the antenna via a supply cable 116 that may extend from the console 102 to the ablation probe 110. The supply cable 116 may also provide a coolant to the ablation probe 110. The coolant may be a saline solution or other coolant that may be supplied to the ablation probe 110 from a coolant receptacle 112 via a pump cartridge 114 or other pump mechanism.

During an ablation treatment, the needle of the ablation probe 110 may be positioned at or near a target tissue in a patient. A power signal may be supplied to the ablation probe 110 from the console 102. The power signal may be supplied to the ablation probe 110 via the supply cable 116. The power signal may cause microwaves to be emitted from an antenna in the needle of the ablation probe 110. The microwaves may cause the tissue at the needle to be heated to a suitable temperature to destroy the target tissue. A temperature sensor 106 may be positioned at or near the target tissue to measure a temperature that may be achieved at or near the ablation zone. In addition and as further described below, the needle or the handle of the ablation probe 110 may include other thermocouples or temperature probes to provide information regarding temperatures of the coolant or the needle.

Referring now to FIG. 2, an example ablation probe 200 is shown. The ablation probe 200 may be used with the microwave ablation apparatus 100 previously described. The ablation probe 200 may include a needle 202, a handle 204, and a supply cable 206. The ablation probe 200 may be a disposable or single-use probe that is used during a single procedure or for a single patient. In other examples, the ablation probe 200 (or portions of the ablation probe 200) may be re-used.

The needle 202 of the ablation probe 200 may be an elongated member that may be inserted into a patient at or near the target tissue. The needle 202 may be a metal, fiberglass, composite, alloy or other rigid material. The needle 202 may be cylindrical and pointed. The needle 202 may be hollow such that various elements are positioned inside the needle 202. For example, a power line and an antenna may be located inside needle 202. The power line may be positioned inside the needle 202 to deliver a power signal from a microwave generator to the antenna. Coolant lines may also be positioned inside the needle to deliver coolant to a distal end of the needle 202. The coolant may be used to control a temperature of the needle 202 and/or to control a size and shape of the ablation zone that may be created at the distal end of the needle 202.

The handle 204 may connect and/or operably couple aspects of the ablation probe 200 that are provided from the supply cable 206 to the needle 202. The supply cable 206 may include a power line that delivers the power signal from the microwave generator. The supply cable 206 may also include a coolant supply line and a coolant return line. The coolant supply line may deliver coolant to the needle 202 and the coolant return line may convey the coolant away from the needle 202 after it has been used to cool the needle 202. The supply cable 206 may also include one or more wires that may provide signals from one or more temperature sensors, thermocouples, flow sensors, or other sensors that may provide information regarding operating characteristics of the ablation probe 200.

The supply cable 206 may be an elongated tube or conduit and each of the power line, the coolant supply line, the coolant return line, and other wires and lines may extend within an outer shell of the supply cable 206. A length of the supply cable 206 may allow a user of the ablation probe 200 to position the needle 202 of the ablation probe 200 as desired and to allow the patient to be moved in and/or out of an imaging device during a treatment. For example, a location of the needle 202 may be determined using CT, MRI, Ultrasound, or other imaging devices before, during, or after treatment is performed. Thus, the supply cable 206 may be sufficiently long to allow these diagnostic procedures to be performed. In some examples, the supply cable 206 is at least 12 feet in length. In other examples, the supply cable 206 is at least 10 feet in length. In other examples, other lengths may be used.

The handle 204 may connect and/or operably couple the supply cable 206 to the needle 202. The power signal, the coolant, and/or sensor signals may be conveyed or delivered from the supply cable 206 to the needle 202 through the handle 204. The handle 204 may include a housing that may enclose various aspects such as fluid connections, electrical connections, electrical components, and the like. The handle 204 may be configured to orient the needle 202 in a desired alignment relative to the longitudinal axis of the supply cable 206. In the example shown, the handle 204 is a right-angle handle. The handle 204 orients the needle 202 at about 90 degrees (or substantially perpendicular) to the longitudinal axis of the supply cable 206. In other examples, the handle 204 may orient the needle 202 at a different angle relative to the longitudinal axis of the supply cable 206.

Turning now to FIG. 3, an example needle 202 is shown. The needle 202 may include a shell 302, a tip 304, a power cable 306, a choke 308, and a coolant conduit 310. During a treatment, a microwave power signal may travel to an antenna 312 along the power cable 306 from a microwave generator. The power cable 306 may be a coaxial cable that can include an outer conductor and an inner conductor. The current that is passed to the antenna 312 may bounce back or be reflected back (in a direction away from the tip 304) along the outer conductor of the power cable 306. This back current is undesirable because the back-current may cause the ablation zone that is created at the needle 202 to become elongated or have a tear-drop shape rather than being more spherical in shape. The elongated ablation zone may radiate or heat healthy tissues adjacent or near the target tissues. It is desirable, therefore, to prevent and/or minimize distortions that may occur to the size and/or shape of the ablation zone due to reflected to back-current.

The choke 308 may be provided in the needle 202 to prevent, reduce, or minimize back-current that travels back along the power cable 306 in a direction away from the tip 304. The choke 308, as further described below, may include a conductive outer casing that can be electrically coupled to the outer conductor of the power cable 306 to limit, reduce, and/or minimize current that is reflected back along an outer surface of the power cable 306 away from the antenna 312.

The needle 202 may be elongated in a longitudinal or axial direction along axis 320. The various elements of the needle 202 may be concentrically positioned about the axis 320. The shell 302 may be the outermost member of the needle 202 and may be a hollow cylindrical tube that defines an inner cavity. The outer surface of the shell 302 may have a smooth profile to facilitate insertion of the needle 202 into a patient at the target tissue. The tip 304 may be positioned at the distal end of the shell 302 and may be pointed to further facilitate insertion and positioning of the needle 202.

The shell 302 can be made of multiple materials joined together. A first portion of the shell 302 may be made of a stainless steel or other relatively rigid material to provide structure to the needle 202. A second portion located at or near a distal end of the shell may be made of a non-conductive material such as a plastic, fiberglass, ceramic, composite or other non-metallic material. The second portion is made of suitable non-conductive material so as to allow RF energy to be conveyed from the antenna 312 through the shell 302. The end portion may, therefore, be located at a portion of the needle 202 at or near the antenna 312 which is typically located at or near the tip 304.

The cable 306 may be positioned as the innermost element of the needle 202 and may be positioned in a center of the needle along the axis 320. The cable 306 is configured to deliver a suitable current from a microwave generator (not shown) to cause microwave energy (or other RF energy) to be emitted from the antenna 312. The cable 306 can be a coaxial cable that includes a center conductor and an outer conductor. The center conductor may be positioned at a central axis of the cable 306. The outer conductor may be separated from the inner conductor and be configured as a cylinder of conductive material positioned radially outward of the inner conductor along the length of the cable 306.

The antenna 312 may be positioned at a distal end of the cable 306 at or near the tip 304 of the needle 202. The antenna 312 can be configured in any suitable manner such as monopole antenna or a dipole antenna. While one type or configuration of the antenna 312 may be shown in the figures, it should be appreciated that other types or configurations can also be used. As previously described, the antenna 312 is configured to emit RF or microwave energy to heat tissue that is located at or near the antenna 312. In this manner, the needle 202 can generate an ablation zone that is generally located around the antenna 312 at or near the tip 304 of the needle 202. The end of the antenna 312 may be supported by a ferrule 314. The ferrule 314 may be a cylindrical elongated member that includes an inner hole positioned at the center. The antenna 312 may be received into the inner hole of the ferrule. The antenna 312 may be supported in a central location in the shell 302 by the ferrule 314. The ferrule 314 may be coupled to the cable 306 and/or the antenna 312 and be supported on an opposite end at the tip 304. The ferrule 314 may position and maintain the antenna 312 in a desired position radially at the center or axis of the shell 302. The ferrule 314 may also position and maintain the antenna 312 in a desired axial position relative the tip 304.

When in use, the energy of the antenna 312 and current reflected along the outer conductor of the cable 306 may cause the needle 202 to increase in temperature. The heating of the needle 202 may cause the heating zone to become elongated in an axial or longitudinal direction of the needle 202. The elongated heating zone may have an elliptical or teardrop shape, for example. In addition, if the exterior surface of the shell 302 increases in temperature, the tissue in contact with the shell 302 may stick to the shell 302. When the needle 202 is retracted after treatment, the tissue may tear resulting in tearing and/or bleeding that is undesirable. To prevent undesirable heating of the needle 202 and/or to maintain a more spherical ablation zone shape, the needle 202 may include a cooling system to actively cool the needle 202 during operation.

In this example, the needle 202 includes the coolant conduit 310. The coolant conduit 310 can be located between the cable 306 and the shell 302. Thus, the coolant conduit 310 can be located radially outward of the cable 306 and radially inward of the shell 302. The coolant conduit 310 extends longitudinally into the needle 202 along the central axis 320 with a terminating end 316 of the coolant conduit 310 being positioned at or near the antenna 312.

The coolant conduit 310 is configured to deliver a cooling fluid toward the tip 304 of the needle 202. While not shown, the cooling system can include a source of cooling fluid and/or a suitable pump that can deliver a flow of cooling fluid in the interior of the coolant conduit 310 toward the tip 304 of the needle 202. The cooling fluid may return to the source of the cooling fluid or be exhausted or otherwise deposited to another location. During operation, the cooling fluid can flow in a flow path in which the cooling fluid is delivered to the needle in an input flow path defined by an interior surface of the coolant conduit 310 and an exterior surface of the cable 306. The cooling fluid may flow away from the tip 304 in a return flow path defined by an exterior or outer surface of the coolant conduit 310 and an interior surface of the shell 302. The cooling fluid may be any suitable flowable cooling liquid or gas such as water, saline, carbon dioxide gas, or the like.

Referring now to FIG. 4, the example coolant conduit 310 is shown. The example coolant conduit 310 may include a distal portion 404 and a proximal portion 406. The distal portion 404 may be positioned closer to the tip 304 while the proximal portion 406 may be positioned closer to the handle 204 (not shown). The distal portion 404 and the proximal portion 406 of the coolant conduit 310 may be made of different materials. In one example, the proximal portion 406 may be made of a metal or alloy tube. The proximal portion 406 may be made of a stainless steel, for example. The proximal portion 406 may provide rigidity to the needle 202. The distal portion 404 may be made of a non-conductive material or non-metallic material such as a plastic, composite, fiberglass, polyimide, or the like. In the example shown, the distal portion 404 may be made of a polyimide material. In other examples, the distal portion 404 may be made of a fiberglass material that may be made of one or more layers of fibers. The fibers may be oriented in different directions in the layers of the fiberglass to add strength and rigidity to the distal portion 404. The distal portion 404 may be made of a non-conductive or non-metallic material such that the distal portion 404 does not interfere with the microwaves that are emitted by the antenna 312 during use of the ablation probe 200. The material of distal portion 404 is preferably a heat resistant material to withstand the temperatures that may be achieved during a microwave ablation procedure.

The distal portion 404 may be joined to the proximal portion 406 using various joining processes or structures. In one example, the inner diameter of the distal portion 404 may be slightly larger than the outer diameter of the proximal portion 406. The distal portion 404 may be fit over the proximal portion 406 for a predetermined joining region 410. The distal portion 404 may be fixed and/or connected to the proximal portion 406 using an adhesive, epoxy, or other joining material. In other examples, the distal portion 404 may be fixed and/or connected to the proximal portion 406 using staking, welding, or other joining process.

The distal portion 404 and the proximal portion 406 may define a continuous flow path for the coolant into the needle 202. The cable 306 may be positioned inside the proximal portion 406 and the distal portion 404 and define a supply flow path for the coolant. The coolant may flow in a direction toward the tip 304 through the space defined by the inner surfaces of the distal portion 404 and the proximal portion 406. The coolant may then flow out of the coolant conduit 310 at a distal end 408 of the distal portion 404. The coolant may then flow in an opposite direction away from the tip 304 in the coolant return flow path defined by the inner surface of the shell 302 and the outer surfaces of the distal portion 404 and the proximal portion 406.

The distal end 408 of the distal portion 404 is positioned at or near the antenna 312. The arrangement and the relative distance of the distal end 408 of the distal portion 404 from the tip 304 may determine a size and shape of the ablation zone that is created during an ablation procedure. The flow rate and the flow path of the coolant may be used to circulate the coolant so as to achieve a desired size and shape of the ablation zone.

It is possible that during manufacture or assembly of the ablation probe 200, that the coolant conduit 310 may move or shift. For example, if the distal portion 404 is not adequately fixed to the proximal portion 406, the amount of overlap between the distal portion 404 and the proximal portion 406 may change after assembly. If the distal portion 404 were to move toward the tip 304, the flow path of the coolant may become obstructed. To prevent this and/or to minimize any disruption to the flow of coolant, the coolant conduit 310 may include an angled end surface 410. If the coolant conduit 310 were to have a straight end surface (i.e., if the end surface of the distal portion 404 were to be oriented substantially perpendicular to the axis of the coolant conduit 31), the coolant could be blocked or restricted if or when the end surface of the coolant conduit 310 shifted toward the tip 304 and became located in close axial proximity to or in contact with the tip 304 inside shell 302 (see FIG. 3). To mitigate against this possibility, the end surface 410 of the coolant conduit 310 is cut, formed, or otherwise shaped to be angled relative the axis of the coolant conduit 310. With such a shape, even if the coolant conduit 310 were to move axially toward the tip 304 and even contact the tip 304, coolant would be able to flow out of the distal end 408 of the distal portion 404.

Another example of a coolant conduit 500 is shown in FIG. 5. The distal end 504 of the distal portion 502 of coolant conduit 500 may include an angled end surface. In this example, the end surface may be angled at angle A relative to axis 506. In one example, angle A may be about 45 degrees. In another example, angle A may be about 30 degrees. In still other examples, the angled end surface may be oriented at other angles relative to the axis 506.

Another example coolant conduit 600 is shown in FIG. 6. In this example, the distal end 604 of distal portion 602 of coolant conduit 600 may include a pointed end surface. In this example, the distal end 604 may include multiple angled end surfaces that meet at a point. The end surfaces of the distal end 604 may be oriented at angle B and angle C relative to axis 606. The angles B and C may be substantially the same in some examples. In other examples, the angles B and C may be different. In one example, the angles B and C are substantially the same and may be about 45 degrees. In another example, the angle B and C are substantially the same and may be about 30 degrees. In other examples, other angles may be used.

The end surface of the coolant conduit 310 may have end surfaces with profiles different from those shown in FIGS. 3, 4, 5 and/or 6. In other examples, the end surface of coolant conduit 310 may have a rounded shape, an inward V-cut shape, a multi-pointed shape, or other shapes. The end surface of the coolant conduit may have an end surface that is not parallel to a surface of the tip 304 that faces toward the end surface of the coolant conduit 310. In still other examples, a distal end of the coolant conduit 310 may have multiple holes through the wall of the conduit to allow coolant to flow laterally out of the conduit in the event the end of the conduit becomes obstructed.

The following is a list of non-limiting illustrative embodiments disclosed herein:

Illustrative embodiment 1: A microwave ablation probe comprising: a shell extending in an axial direction and defining an inner cavity; a tip positioned on a distal end of the shell; a cable comprising a microwave antenna extending axially in the inner cavity; and a coolant conduit positioned radially outward of the cable defining a coolant flow path toward the tip, a distal end of the coolant conduit including an angled end surface oriented at an angle relative to an axis of the coolant conduit.

Illustrative embodiment 2: The microwave ablation probe of illustrative embodiment 1, wherein the coolant conduit comprises a distal portion connected to a proximal portion, the distal portion comprising a different material from the proximal portion and terminating at the angled end surface.

Illustrative embodiment 3: The microwave ablation probe of any of illustrative embodiments 1 or 2, wherein the proximal portion comprises a metal material.

Illustrative embodiment 4. The microwave ablation probe of any of illustrative embodiments 1 to 3, wherein the distal portion comprises a polyimide material.

Illustrative embodiment 5. The microwave ablation probe of any of illustrative embodiments 1 to 4, wherein the coolant flow path toward the tip is located inside the coolant conduit.

Illustrative embodiment 6. The microwave ablation probe of any of illustrative embodiments 1 to 5, wherein the angled end surface is not parallel to an end surface of the tip that faces the coolant conduit in the inner cavity.

Illustrative embodiment 7. The microwave ablation probe of any of illustrative embodiments 1 to 6, wherein distal end of the coolant conduit is positioned at or near the antenna in the inner cavity.

Illustrative embodiment 8. The microwave ablation probe of any of illustrative embodiments 1 to 7, wherein the angled end surface defines a planar surface.

Illustrative embodiment 9. The microwave ablation probe of any of illustrative embodiments 1 to 8, wherein the angled end surface defines a pointed end of the coolant conduit.

Illustrative embodiment 10. The microwave ablation probe of any of illustrative embodiments 1 to 9, wherein the angled end surface defines a pointed end located at a center axis of the coolant conduit.

Illustrative embodiment 11. A coolant tube for use in a microwave ablation probe comprising: a proximal portion; and a distal portion connected to the proximal portion, wherein the proximal portion and the distal portion define a continuous coolant flow path, a distal end of the distal portion comprising an angled end surface.

Illustrative embodiment 12. The coolant conduit of illustrative embodiment 11, wherein the proximal portion comprises a metal material.

Illustrative embodiment 13. The coolant conduit of any of illustrative embodiments 11 or 12, wherein the distal portion comprises a polyimide material.

Illustrative embodiment 14. The coolant conduit of any of illustrative embodiments 11 to 13, wherein the angled end surface defines a planar surface.

Illustrative embodiment 15. The coolant conduit of any of illustrative embodiments 11 to 14, wherein the angled end surface defines a pointed end of the coolant conduit.

Illustrative embodiment 16. The coolant conduit of any of illustrative embodiments 11 to 15, wherein the angled end surface defines a pointed end located at a center axis of the coolant conduit.

Illustrative embodiment 17. A microwave ablation apparatus comprising: an ablation console comprising a microwave generator; and a microwave ablation probe coupled to the ablation console, the microwave ablation probe comprising: a shell extending in an axial direction and defining an inner cavity; a tip positioned on a distal end of the shell; a cable comprising a microwave antenna extending axially in the inner cavity; and a coolant conduit positioned radially outward of the cable defining a coolant flow path toward the tip, a distal end of the coolant conduit including an angled end surface oriented at an angle relative to an axis of the coolant conduit.

The foregoing is provided for purposes of illustrating, explaining, and describing embodiments of these disclosures. Modifications and adaptations to these embodiments will be apparent to those skilled in the art and may be made without departing from the scope or spirit of these disclosures.

What is claimed is:

1. A microwave ablation probe comprising:
a shell extending in an axial direction and defining an inner cavity;
a tip positioned on a distal end of the shell;
a cable comprising a microwave antenna extending axially in the inner cavity; and
a coolant conduit positioned radially outward of the cable defining a coolant flow path toward the tip, a distal end of the coolant conduit including an angled end surface oriented at an angle relative to an axis of the coolant conduit.

2. The microwave ablation probe of claim 1, wherein the coolant conduit comprises a distal portion connected to a proximal portion, the distal portion comprising a different material from the proximal portion and terminating at the angled end surface.

3. The microwave ablation probe of claim 2, wherein the proximal portion comprises a metal material.

4. The microwave ablation probe of claim 3, wherein the distal portion comprises a polyimide material.

5. The microwave ablation probe of claim 1, wherein the coolant flow path toward the tip is located inside the coolant conduit.

6. The microwave ablation probe of claim 1, wherein the angled end surface is not parallel to an end surface of the tip that faces the coolant conduit in the inner cavity.

7. The microwave ablation probe of claim 1, wherein distal end of the coolant conduit is positioned at or near the antenna in the inner cavity.

8. The microwave ablation probe of claim 1, wherein the angled end surface defines a planar surface.

9. The microwave ablation probe of claim 1, wherein the angled end surface defines a pointed end of the coolant conduit.

10. The microwave ablation probe of claim 1, wherein the angled end surface defines a pointed end located at a center axis of the coolant conduit.

11. A coolant tube for use in a microwave ablation probe comprising:
a proximal portion; and
a distal portion connected to the proximal portion,
wherein the proximal portion and the distal portion define a continuous coolant flow path, a distal end of the distal portion comprising an angled end surface.

12. The coolant conduit of claim 11, wherein the proximal portion comprises a metal material.

13. The coolant conduit of claim 12, wherein the distal portion comprises a polyimide material.

14. The coolant conduit of claim 11, wherein the angled end surface defines a planar surface.

15. The coolant conduit of claim 11, wherein the angled end surface defines a pointed end of the coolant conduit.

16. The coolant conduit of claim 11, wherein the angled end surface defines a pointed end located at a center axis of the coolant conduit.

17. A microwave ablation apparatus comprising:
an ablation console comprising a microwave generator; and
a microwave ablation probe coupled to the ablation console, the microwave ablation probe comprising:
a shell extending in an axial direction and defining an inner cavity;
a tip positioned on a distal end of the shell;
a cable comprising a microwave antenna extending axially in the inner cavity; and
a coolant conduit positioned radially outward of the cable defining a coolant flow path toward the tip, a distal end of the coolant conduit including an angled end surface oriented at an angle relative to an axis of the coolant conduit.
